(19) United States
(12) Patent Application Publication
(10) Pub. No.: US 2011/0030122 A1
(43) Pub. Date: Feb. 10, 2011

(54) Orthopedic Hand Glove

(75) Inventor: Carlos E. Capurro, Pompano Beach, FL (US)

Correspondence Address: Carlos E. Capurro, 1164 SW 4th Terrace, Pompano Beach, FL, 33060, US

(73) Assignee: CARLOS ENRIQUE CAPURRO, Pompano Beach, FL (US)

(21) Appl. No.: 12/462,760

(22) Filed: Aug. 10, 2009

(51) Int. Cl. A41D 19/00; A61F 5/00

(52) U.S. Cl. 2/162; 2/163; 2/167; 602/21

(57) ABSTRACT

This invention provides a novel glove for both left and right hands presenting properties for a unique design apparatus related to the orthopedic field. A glove, in particular for helping the hand avoid doing involuntary folding of fingers, griping or tightening of the hand into a fist position due to possible health ailments or physical disorders. This is accomplish by placing on the palm side of the glove four external supportive ribs made out of flat aluminum filaments that extend from the base of the palm to the top of the fingers. By four fingers it is meant the index, the middle, the ring and the little fingers.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

This invention relates in general to the field of gloves and more specifically to gloves made of synthetic knit fabric or other stretchable materials. Field of the invention relates to improved gloves and other similar articles of wearing apparels pertaining to the health field more specific to the orthopedic field and similar fields that work to find treatments and solution to diseases or physical disorders of the human hands more specifically of the fingers.

Some individuals tend to show degenerative health issues pertaining the joints or articulation of the fingers, the consequences could result on certain interferes in the normal functioning of the hands. The individuals with this health issue are prone to experience an involuntary action in the form of closing of their hands forming a fist, leading in time not only to experience discomfort and pain but inability to maintain or return the hand to a normal open position. Although this problem can be seen more in the elderly individuals it can also occur on younger adults in a less manner. This orthopedic glove is aimed to provide the users an innovative and simple way of dealing with this particular health issue by means of wearing this particular glove.

A secondary field of this invention is related to gloves and, more specifically to orthopedic hand gloves showing for improvement of fabrications and vanguard of design by using a method for combining materials such as stretchable and flexible garment materials complementing it with flat aluminum filaments used for the support ribs.

### BRIEF SUMMARY OF THE INVENTION

The main objective of the present invention is to contribute and provide a unique and new simple solution to the involuntary folding, griping, tightening of the hand into a fist position due to ailments or physical disorders. This solution comes in the form of the assemblage of an orthopedic glove for both hands in which special ribs structures provide support to the four fingers (namely the index, the middle, the ring and the little finger) unable the fingers to fold forming a tight grip thus causing pain and atrophy of the hand of users.

The way the glove works is such that when the user's hand wants or starts to fold in an involuntary manner, trying to form a fist type position, the ribs structure inside the glove will not allow this closing effort; this orthopedic glove is aimed to stop such involuntary process and hopefully alleviate some of the pain involved thus slowing down or in the best case stop any degenerative process that might be taking place.

Another objective is to provide improvement of fabrications of gloves which includes the method of construction and combination of materials as described in the claim.

The main advantage of this invention is that the user will experience through a simple way relief from the pain from using the glove.

Another significant advantage is the ability for the user to flex and bend the flat aluminum filaments that form the supporting ribs to the desire curve position the user feels to be more comfortable.

Another advantage is its easy wear or usage as it can be carried anywhere the user goes, also to consider the glove can be use the amount of time desired in a comfortable manner as the glove provides a breathable, stretching material for a garment suitable for use in varying environments. If the user becomes tired or uncomfortable at using it all it takes is to remove it from the hands. It is reusable as many times as the user wants as long as the user takes basic well care of it and removes the laminated ribs when washing the glove.

### BRIEF DESCRIPTION OF THE SEVERAL VIEWS OF THE DRAWING

The accompanying figures show on detail description different views of the same hand glove in accordance with the embodiment of the present invention:

FIG. 1 shows a dual view of the same orthopedic glove, one being the palm side view and the other being the backhand view. A close up of the palm side shows how the flat aluminum filaments fit inside the pockets to form the rib through the opening of this pockets at the bottom of the base of the palm.

FIG. 2 is a side hand view of the orthopedic glove that allows the viewer to see the elongating supportive ribs at a slight bending position to represent the pressure from the user's finger if as the fingers would be trying to close into forming a fist.

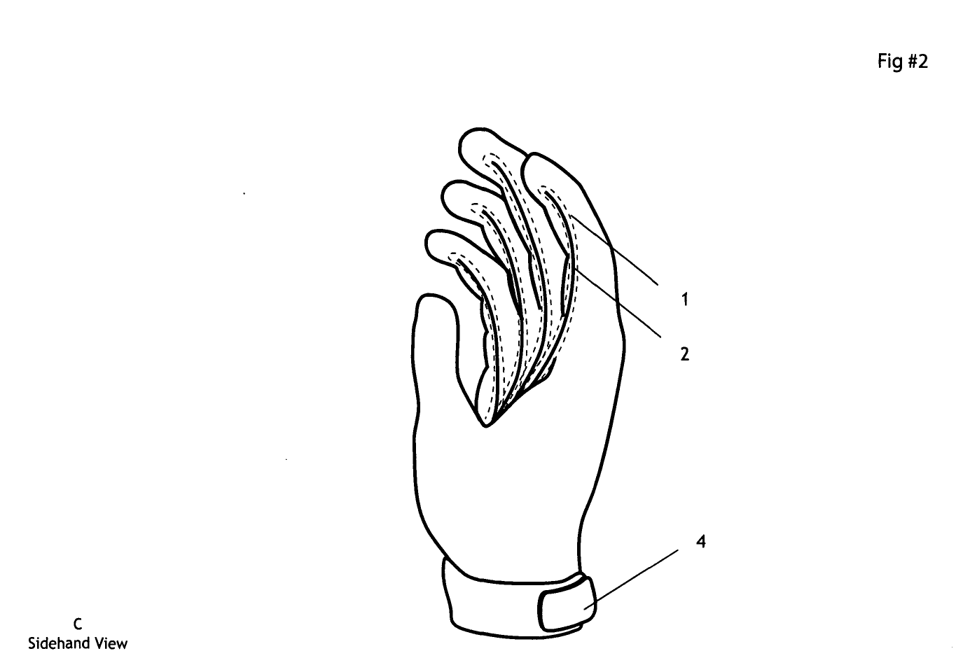

FIG. 3 is a rearward cross section view of FIG. 1 shows the area of embodiment for the hand. Also allows a detail review on such angle of the opening for the four rib's pocket where the flat aluminum filaments would slide in, these four openings being equally located at level of the base of the palm.

FIG. 4 is a multiple view of the same glove in accordance with the embodiment of the present invention.

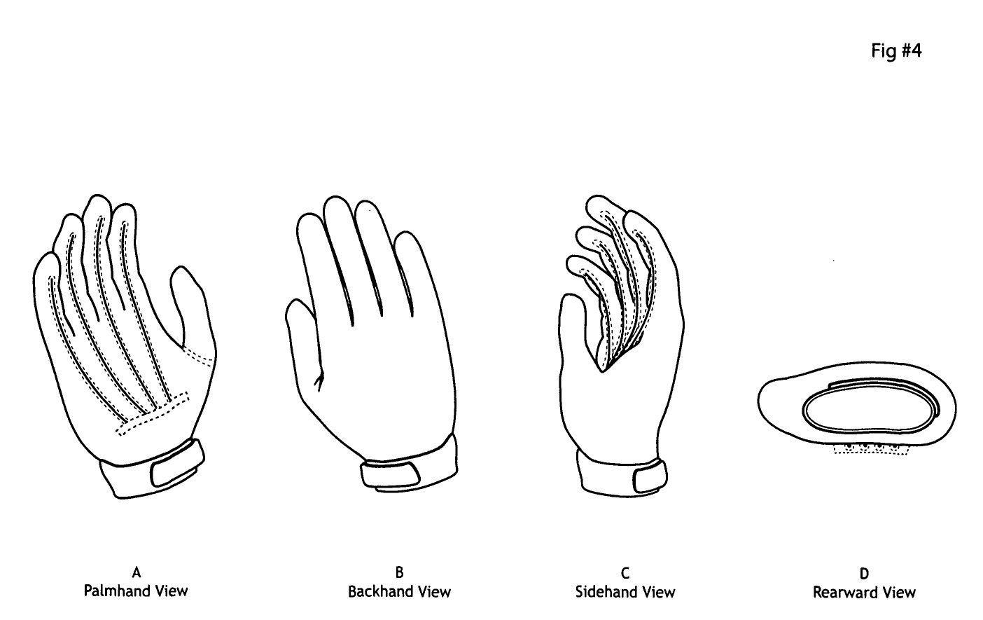

### DETAILED DESCRIPTION OF THE INVENTION

The object of the present invention is to provide a unique solution to a problem of involuntary folding, griping or tightening of the hand into a fist position due to ailments or physical disorders. With further reference to the drawings provided

About the Making:

A glove comprising an interior and an exterior side both having a perimeter corresponding to the shape of a hand, the perimeter defined approximately by ulnar and radial sides, a phalangeal portion, a metacarpal portion and a cuff portion.

A glove compromising a palm side and a back side stitched together so as to form an embodiment for the holding of the hand, more specific receiving a phalangeal portion, a metacarpal portion and a cuff portion corresponding to the phalangeal, metarcarpal and wrist of a human hand. A thumb portion may secure to the metacarpal portions.

A glove constructed of a stretching and breathable material having substantially uniform thickness, such material as nylon polyester based and other based synthetic knit fabric, more specific to be dry fit with Lycra jersey with hydrophilic polyester elastin. This material allows for an effective skin tight fit about the user's hand in a comfortable manner.

A glove compromising a cuff area embodies the wrist of a user, on the exterior wherein around the wrist there is a “wrist attaching strap” to adjust and secures in position the glove to the hand, more specific this strap having on the tip end a Velcro material for such purpose. This cuff area being double folded showing for a slightly thicker tactility than the rest of the glove being but being of the same material of the rest of the glove, more specific this material to be a dry fit with lycra jersey with hydrophilic polyester elastane.

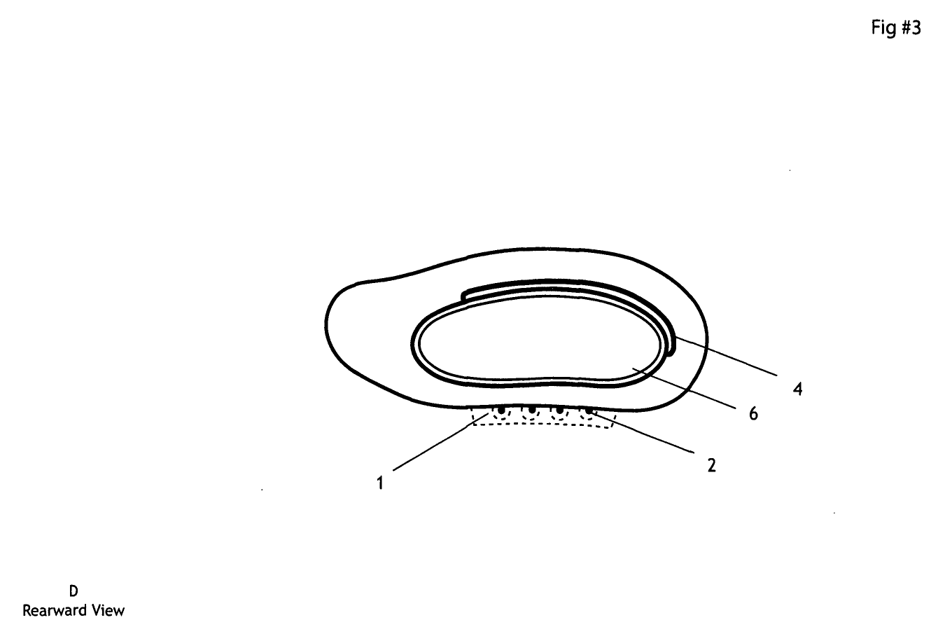

As shown in FIG. 3 the embodiment of the glove in where the palm (metacarphal) section fits 6 when looked from the rearward view has a generally oblong opening which is typically elliptical in shape in use. It preferentially has and arcuate shape similar to an open mouth. In such systems the opening formed by the cuff tends to remain closed inasmuch as the fabric tends to collapse the opening. This is especially true of gloves formed of more flexible materials such as synthetic knit fabrics and other stretchable materials. Such materials have virtually no ability to retain their shape, and users must therefore expand the opening before inserting the hand.

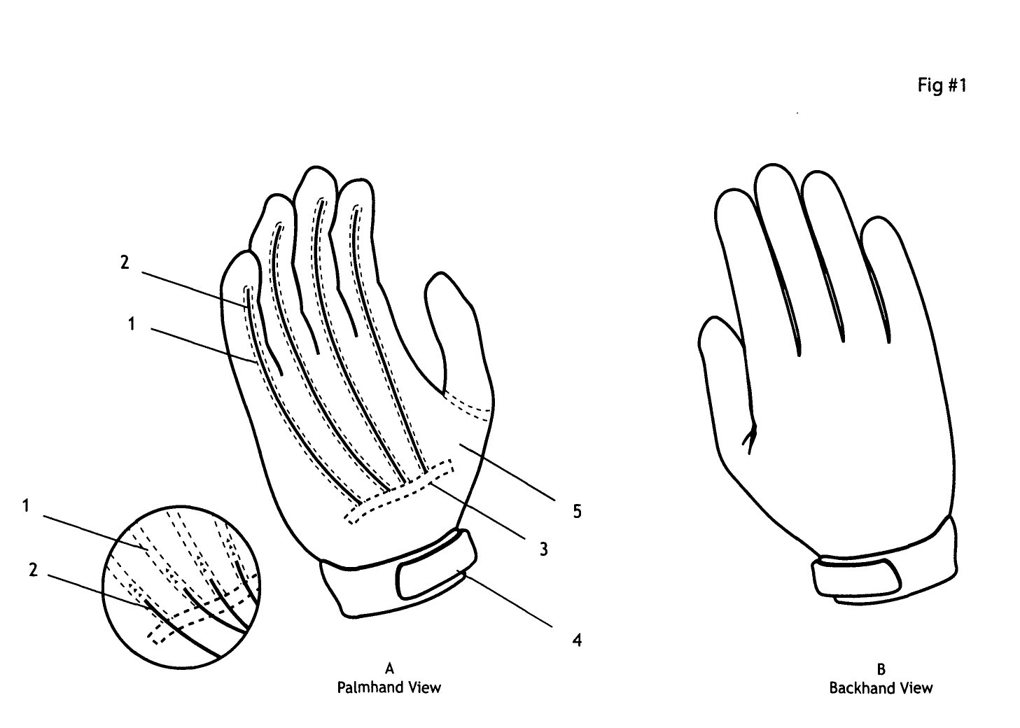

As shown in FIG. 1 a glove also compromising a portion formed by four longitudinal pockets 1 that are seamed on the surface of the palm side 5, the purpose of this pockets 1 are to carry in the inside the flat aluminum filaments 2 that compose the supportive ribs 2. This pockets 1 are made of the same flexible material than the rest of the glove but has a thicker composition considering that are meant to carry in the inside the aluminum filaments that make the ribs 2. This four rib's compartment pockets projects longitudinal starting at the base of the palm (distal phalanges) and ending over the so called dip joint point of the four fingers, more specific the index, the middle, the ring and the little fingers. Each of the pockets has openings located at the end located at the base of the palm sector where a welt 3 is sewed across them at the opening level for the sole purpose of functions as retainer or holder unable for the flat aluminum ribs 2 to slip out of position and fall out. This aluminum filament or supportive ribs 1 are evenly flat and narrow proportional to ⅓ of the finger width. These supportive aluminum ribs 1 are flexible allowing for manual bending with certain amount of pressure and they can be easily removed from the pockets if necessary.

All the materials of the glove are water resistant.

About the Usage:

The supportive rib filaments, unless if the user decides to watch the glove in water, should always be kept safe at all the time positioned inside their individual pockets. In general the proper usage is similar to wearing any normal garment glove, as seen in FIG. 3 the user introduce the hand through the oblong opening 6 into it until each finger is properly in place on all the finger stalls. With the hand properly inside the glove needs to be tightened and secure by adjusting the “wrist attaching strap” 4 on the cuff part of the glove. Once the hand is in placed the user manipulates manually the ribs to the set angle position desired for the period of time the user wants. In addition it is desirable that the glove is highly pliable and flexible to enable the user to use the hand normally because of the position of the supportive ribs there could be certain limitation on the normal usage of the hand while using the gloves. Upon user discretion of the time spent with the glove the user can remove the glove from the hand. Although it is important to note that the amount of time to wear the glove should be in relation with the seriousness of the ailment the user might experience, there is no limit on timeframes of usage.

1. A hand covering glove garment of elastic and flexible materials with attached elongating supportive ribs placed on the external side of the palm area.

2. Referring to claim 1 wherein said “elongating supportive ribs” extend on one end from the lower base of the palm, more specifically the hamate and capitate bones; to the tip of the finger, more specifically the distal phalanges just over the dip joint point of the fingers. By fingers more specifically the index finger, middle finger, ring finger, little finger.

3. Referring to claim 1 and 2 wherein said “elongating supportive ribs” made out each of flat aluminum filaments each individually placed inside a sewed stretchable knitted pockets.

4. A hand covering glove according to claim 1 wherein the glove being made of stretchable and flexible fabric such as nylon polyester based and other based synthetic knit fabric, more specific to be dry fit with Lycra jersey with hydrophilic polyester elastin.

5. A hand covering glove according to claim 1 wherein around the wrist there is a “wrist attaching strap” to adjust and secures in position the glove to the hand.

6. A hand covering glove according to claim 1 a breathable and suitable material for a garment which is easily fabricated and is suitable for use in varying environments

7. A hand covering glove according to claim 1 showing an improvement of fabrications of gloves which includes the method of combining materials such as using garment stretching materials and flat aluminum material for the ribs

8. A hand covering glove according to claim 1 in particular for the treatment of arthritis or degenerative ailments of joints, articulations and bones in the hand more specifically the fingers that could result in pain and or deformation of the hand and more specifically the fingers producing the inability of a proper use of the hands.

9. A hand covering glove according to claim 1 an ornamental design for a unique orthopedic hand glove for both right and left hand. The embodiments of the invention in which an exclusive property or privilege is claimed are defined as shown in the drawings. A glove, in particular for helping the hand avoid doing involuntary folding of fingers, griping resulting in the tightening of the hand into a fist position causing pain and discomfort.
